(19) United States
(12) Patent Application Publication
(10) Pub. No.: US 2024/0175087 A1
(43) Pub. Date: May 30, 2024

(54) METHODS AND SYSTEMS FOR PREDICTING CANCER HOMOLOGOUS RECOMBINATION PATHWAY DEFICIENCY, AND DETERMINING TREATMENT RESPONSE

(71) Applicant: Sema4 OpCo, Inc., Stamford, CT (US)

(72) Inventors: Eunjee Lee, Stamford, CT (US); Jun Zhu, Stamford, CT (US)

(73) Assignee: Sema4 OpCo, Inc., Stamford, CT (US)

(21) Appl. No.: 18/059,630

(22) Filed: Nov. 29, 2022

(51) Int. Cl. C12Q 1/6886; G16B 20/00

(52) U.S. Cl. CPC C12Q 1/6886; G16B 20/00; C12Q 2600/106; C12Q 2600/112; C12Q 2600/156; C12Q 2600/158

(57) ABSTRACT

A method (100) for providing a homologous recombination DNA repair deficiency (HRD) score for a cancer patient, comprising: receiving (120) information about the cancer patient, the information comprising at least mRNA expression data obtained from a tumor of the cancer patient; analyzing (130), using a trained HRD score model, the received information about the cancer patient to generate an HRD score for the cancer patient; and providing (140), via a user interface, the generated HRD score for the cancer patient.

### FIELD OF THE DISCLOSURE

The present disclosure is directed generally to methods and systems for providing a homologous recombination DNA repair deficiency (HRD) score for a cancer patient.

### BACKGROUND

Breast cancer is the most common and deadly cancer for women, with 12% of US women developing invasive breast cancer over the course of their lifetime. About 15% of invasive breast cancer cases are triple negative breast cancer (TNBC) cases, which are characterized by lack of expression of estrogen receptor (ER), progesterone receptor (PR) and human epidermal growth factor receptor type 2 (HER2). TNBC is a heterogeneous disease with distinct molecular subtypes that differentially associate with aggressive behavior and prognosis and differentially respond to chemotherapy and targeted agents.

Currently, taxol-based chemotherapy is one of the central pillars of TNBC treatment. However, only a small fraction of TNBC patients respond to chemotherapy. While new treatment options, such as poly ADP ribose polymerase (PARP) inhibitors, immunotherapy, and a combination with platinum-based chemotherapy are available for TNBC patients, potential responders to these new treatments are not yet clearly defined. Therefore, it is critical to stratify TNBC tumors who may benefit from the new treatment options.

Both BRCA1 and BRCA2 are crucial for the process of DNA repair by homologous recombination (HR), which are largely involved in the repair of DNA lesions that stall DNA replication forks and/or cause DNA double-strand breaks (DSBs). BRCA1- and BRCA2-null tumors are thus deficient in HR and are selectively sensitive to compounds that increase the demand on HR, such as platinum-based chemotherapy and poly ADP ribose polymerase (PARP) inhibitors. The inability to perform HR-dependent DSB repair ultimately leads to tumor cell death. Indeed, preclinical studies and Phase I/II clinical trials have shown that BRCA1- and BRCA2-mutation carriers have a high sensitivity to PARP inhibitors.

Breast cancer patients with a BRCA1 germline mutation are more likely to have a triple receptor negative phenotype. Moreover, some sporadic TNBCs often share traits with familial-BRCA cancer including harboring DNA repair defects. Previous studies postulated that sporadic TNBCs have diverse defects in HR-dependent DSB repair, through somatic mutations in BRCA1 and BRCA2, promoter methylation of BRCA1 and RAD51C, and other as yet to be identified mechanisms, suggesting a potential of PARP inhibitor treatment for TNBC. However, currently, PARP inhibitor therapy is approved only for TNBC patients with a germline BRCA mutation. Several clinical trials indicate that PARP inhibitor use for sporadic TNBC patients does not have definitive efficacy. Therefore, it is critical to develop a predictive biomarker to identify TNBC patients who may benefit from PARP inhibitor therapy, especially who carry wildtype BRCA1/2 but are deficient for HR-dependent DNA repair pathways.

A predictive biomarker for PARP inhibitor sensitivity would be helpful to personalize the use of PARP inhibitors and/or platinum-based chemotherapy so that patient outcome can be improved. Recent advances in sequencing technologies, such as whole-genome sequencing (WGS), have facilitated to predict homologous recombination DNA repair deficiency (HRD) based on mutational signatures. Analysis of breast cancers WGS data showed that HRD is associated with distinct mutational signatures, i.e. Signature 3 (Sig3). The subsequent study analyzed the association between Sig3 and multi-dimensional events in HR pathway components. Multiple HRD prediction models have been developed including a weighted lasso logistic regression model of mutational signatures called HRDetect and a computational model, Signature Multivariate Analysis (SigMA), that also can be used with low mutation counts. The Myriad myChoice model predicts HRD status using a genomic instability score, i.e. genomic scar, measured through single nucleotide polymorphism (SNP) analysis. Genomic scar is determined by three chromosomal aberrant events including the number of telomeric allelic imbalances (NtAI), loss of heterozygosity score (LOH), and large scale transition (LST). However, genomic signature-based approaches, e.g. mutational signatures and genomic scars, to estimate HRD in tumors has limitations. Mutational signatures, which are readout of the DNA damage and DNA repair processes that have occurred during tumor development, may not reflect the current HRD status in a tumor. For example, secondary somatic mutations that restore BRCA1/2 function can predict resistance to platinum and PARP inhibitors in ovarian cancer. However, the genomic scar patterns do not revert when a tumor has recovered HR function, so they may not be accurate to predict PARP inhibitor sensitivity in patients who progressed on DNA damaging chemotherapy. Therefore, it would be highly beneficial to identify and analyze biomarkers that can reflect current HR pathway functional status.

### SUMMARY OF THE DISCLOSURE

Accordingly, there is a continued need for methods and systems capable of analyzing HR pathway functional status and predicting a cancer patient's homologous recombination DNA repair deficiency (HRD) score.

Various embodiments and implementations are directed to methods and systems for providing a homologous recombination DNA repair deficiency (HRD) score for a cancer patient. An analysis system receives information about the cancer patient, the information comprising at least mRNA expression data obtained from a tumor of the cancer patient. The analysis system analyzes, using a trained HRD score model, the received information about the cancer patient to generate an HRD score for the cancer patient. The analysis system then provides the generated HRD score for the cancer patient to a user via a user interface. When the generated HRD score for the cancer patient indicates that the tumor is HR deficient, the user can implement or administer a treatment to target the HR deficiency, such as chemotherapy and/or a poly ADP ribose polymerase (PARP) inhibitor.

Generally, in one aspect, a method for providing a homologous recombination DNA repair deficiency (HRD) score for a cancer patient is provided. The method includes: receiving information about the cancer patient, the information comprising at least mRNA expression data obtained from a tumor of the cancer patient; analyzing, using a trained HRD score model, the received information about the cancer patient to generate an HRD score for the cancer patient; and providing, via a user interface, the generated HRD score for the cancer patient; wherein the HRD score model is trained by: (i) identifying a plurality of HR pathway genes; (ii) generating a plurality of candidate HR deficiency (HRD) features using (i) DNA mutation data; (ii) DNA copy number variation (CNV) data; (iii) DNA methylation data; and (iv) mRNA expression data to define an activity of each of the plurality of HR pathway genes; (iii) receiving a training dataset comprising records for a plurality of historical cancer patients, at least some of whom were HR deficient; (iv) determining, using the training dataset, a subset of candidate HRD features based on an association between each of the plurality of candidate HRD features and historical cancer patient survival; (v) identifying HRD expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group; (vi) calculating, for each of the plurality of genes for which a HRDES was identified, a distance between the gene and a plurality of HR pathway genes within a constructed molecular causal network for the cancer type; (vii) weighting, based on the calculated distance, one or more of the plurality of genes in the HRDES; and (viii) training, using training dataset, the HRD score model to identify a set of final HRD features and their associated weights.

According to an embodiment, the generated HRD score for the cancer patient indicates that the tumor is HR deficient.

According to an embodiment, the method further includes implementing, when the generated HRD score for the cancer patient indicates that the tumor is HR deficient, a treatment to target the HR deficiency.

According to an embodiment, the treatment to target the HR deficiency is chemotherapy, and/or a poly ADP ribose polymerase (PARP) inhibitor.

According to an embodiment, the set of final HRD features comprises one or more of the genes in TABLE 1.

According to another aspect is a method for treating a cancer patient. The method includes: receiving a generated HRD score for the cancer patient indicating that the tumor is HR deficient; and administering a treatment to the cancer patient; wherein the HRD score is generated by: receiving information about the cancer patient, the information comprising at least mRNA expression data obtained from a tumor of the cancer patient; analyzing, using a trained HRD score model, the received information about the cancer patient to generate an HRD score for the cancer patient; wherein the HRD score model is trained by: (i) identifying a plurality of HR pathway genes; (ii) generating a plurality of candidate HR deficiency (HRD) features using (i) DNA mutation data; (ii) DNA copy number variation (CNV) data; (iii) DNA methylation data; and (iv) mRNA expression data to define an activity of each of the plurality of HR pathway genes; (iii) receiving a training dataset comprising records for a plurality of historical cancer patients, at least some of whom were HR deficient; (iv) determining, using the training dataset, a subset of candidate HRD features based on an association between each of the plurality of candidate HRD features and historical cancer patient survival; (v) identifying HDR expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group; (vi) calculating, for each of the plurality of genes for which a HRDES was identified, a distance between the gene and a plurality of HR pathway genes within a constructed molecular causal network for the cancer type; (vii) weighting, based on the calculated distance, one or more of the plurality of genes in the HRDES is utilized to generate an HR score; (viii) training, using training dataset the HR score model to identify a set of final HRD features and their associated weights.

According to another aspect is a system configured to provide a homologous recombination DNA repair deficiency (HRD) score for a cancer patient. The system includes: information about the cancer patient, the information comprising at least mRNA expression data obtained from a tumor of the breast cancer patient; a trained HRD score model; a processor configured to analyze, using the trained HRD score model, the received information about the cancer patient to generate an HRD score for the cancer patient; and a user interface configured to provide the generated HRD score for the cancer patient; wherein the HRD score model is trained by: (i) identifying a plurality of HR pathway genes; (ii) generating a plurality of candidate HR deficiency (HRD) features using (1) DNA mutation data; (2) DNA copy number variation (CNV) data; (3) DNA methylation data; and (4) mRNA expression data to define an activity of each of the plurality of HR pathway genes; (iii) receiving a training dataset comprising records for a plurality of historical cancer patients, at least some of whom were HR deficient; (iv) determining, using the training dataset, a subset of candidate HRD features based on an association between each of the plurality of candidate HRD features and historical cancer patient survival; (v) identifying HDR expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group; (vi) calculating, for each of the plurality of genes for which a HRDES was identified, a distance between the gene and a plurality of HR pathway genes within a constructed molecular causal network for the cancer type; (vii) weighting, based on the calculated distance, one or more of the plurality of genes in the HRDES is utilized to generate an HR score; and (viii) training, using training dataset the HR score model to identify a set of final HRD features and their associated weights.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating and providing homologous recombination DNA repair deficiency (HRD) score for a cancer patient, in accordance with an embodiment.

FIG. 2 is a schematic representation of a HRD score analysis system, in accordance with an embodiment.

FIG. 3 is a flowchart of a method for training an HRD score algorithm, in accordance with an embodiment.

FIG. 4A is a flowchart of a method for formulating features for HR pathway deficiency predictions, in accordance with an embodiment.

FIG. 4B is a flowchart of a method for defining activity of HR pathway genes, in accordance with an embodiment.

FIG. 5 is a flowchart of a method for generating and providing homologous recombination DNA repair deficiency (HRD) score for a cancer patient, in accordance with an embodiment.

### DETAILED DESCRIPTION OF EMBODIMENTS

The present disclosure describes various embodiments of an HRD score analysis system. More generally, Applicant has recognized and appreciated that it would be beneficial to provide an improved system capable of more accurately analyzing a cancer patient's HR pathway functional status and generating a homologous recombination DNA repair deficiency (HRD) score for the patient. An HRD score analysis system receives information about the cancer patient, the information comprising at least mRNA expression data obtained from a tumor of the cancer patient. The analysis system analyzes, using a trained HRD score model, the received information about the cancer patient to generate an HRD score for the cancer patient. The analysis system then provides the generated HRD score for the cancer patient to a user via a user interface. When the generated HRD score for the cancer patient indicates that the tumor is HR deficient, the user can implement or administer a treatment to target the HR deficiency.

According to an embodiment, the system comprises a computational framework, a NETwork-based Homologous Recombination Deficiency (netHRD), to identify HRD tumors within TNBC by integrating multi-omics data. The model integrates multi-omics data (e.g., DNA mutation, DNA copy number variation, DNA methylation, and mRNA expression) to define activities of HR pathway genes, which could be used to formulate features for determining HR pathway deficiency, giving rise to functional changes in genomic instability, mRNA expression, and tumor microenvironment at the level of a phenotype and, ultimately, responses to chemotherapy and PARP inhibitor therapy. Utilized are TNBC molecular causal networks constructed by integrating multi-omics data, and a network-based HR deficiency prediction model (netHRD) model is developed, aiming to identify HRD tumors that may benefit from chemotherapy and/or PARP inhibitor therapy. The netHRD model is trained on a TNBC dataset (i.e. METABRIC data) and is applied to multiple independent TNBC cohorts treated by chemotherapy. The TNBC tumors with high netHRD scores show significantly better survival or chemotherapy responses compared to tumors with low netHRD scores. Furthermore, it is demonstrated that the netHRD score is associated with PARP inhibitor responses in three independent clinical trials of TNBC cohorts treated with PARP inhibitor in neoadjuvant settings. Taken together, the results demonstrate that the framework definitely identifies patients that will benefit from PARP inhibitor and/or platinum treatment.

According to an embodiment, the HRD score analysis systems and methods described or otherwise envisioned herein provide numerous advantages compared to prior art systems, which are inaccurate and often fail to properly predict or analyze the functional status of the HR pathway and the patient's response to cancer treatment(s). More accurate analysis and prediction of the patient's response to treatment can lead to better treatment and care of the patient, thereby saving lives, and can save the cost of ineffective treatment. Therefore, the HRD score analysis systems and methods described or otherwise envisioned herein reduce costs and improve the care of cancer patients.

The embodiments and implementations disclosed or otherwise envisioned herein can be utilized with any patient care system, including but not limited to clinical decision support tools, patient monitors, and other systems. However, the disclosure is not limited to clinical decision support tools or patient monitors, and thus the embodiments disclosed or otherwise envisioned herein can encompass any device or system capable of performing an HRD score analysis for a cancer patient.

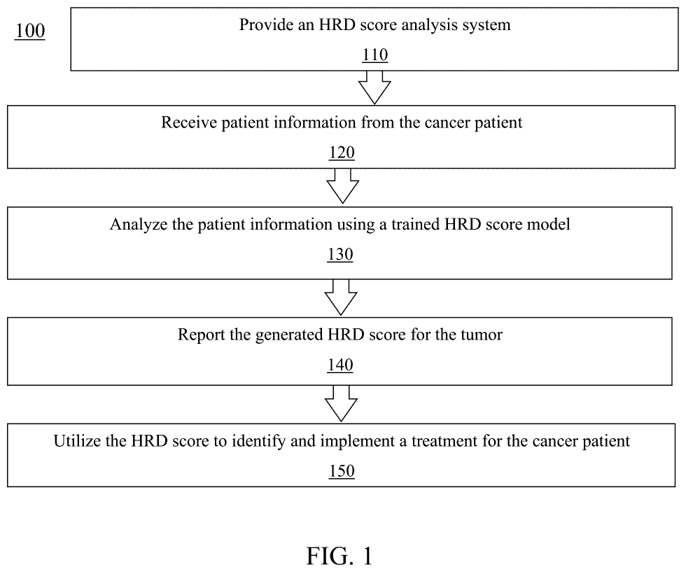

Referring to FIG. 1, in one embodiment, is a flowchart of a method 100 for providing a homologous recombination DNA repair deficiency (HRD) score for a cancer patient, using an HRD score analysis system. The methods described in connection with the figures are provided as examples only, and shall be understood not to limit the scope of the disclosure. The HRD score analysis system can be any of the systems described or otherwise envisioned herein. The HRD score analysis system can be a single system or multiple different systems.

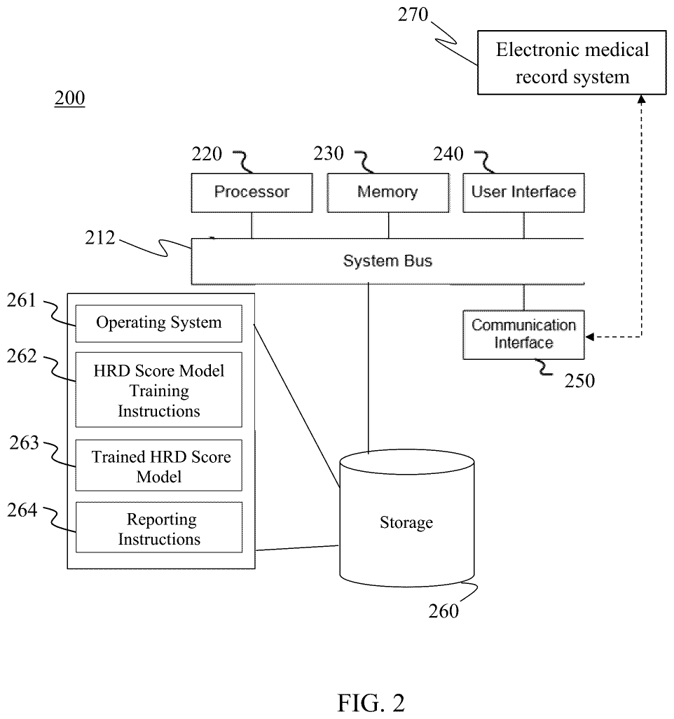

At step 110 of the method, an HRD score analysis system is provided. Referring to an embodiment of an HRD score analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, HRD score analysis system 200 can be any of the systems described or otherwise envisioned herein. Other elements and components of the HRD score analysis system 200 are disclosed and/or envisioned elsewhere herein.

At step 120 of the method, the HRD score analysis system receives information about the patient. The patient information can be any information about the patient that a trained HRD score model can or may utilize for analysis as described or otherwise envisioned herein. According to an embodiment, the patient information comprises at least mRNA expression data obtained from a tumor of the cancer patient. The mRNA expression data can be obtained from the tumor of the patient using any of a variety of methods. For example, the mRNA expression data can be obtained by direct analysis of the mRNA in cells of the tumor, such as RNA-seq. Alternatively, the mRNA expression data can be obtained by indirect analysis of proteins in cells of the tumor. Other methods for mRNA analysis are possible. The mRNA analysis may be an analysis of a sample taken from the tumor, and/or may be an analysis of one or more samples taken from the tumor. The mRNA analysis may be an analysis of a single cell or multiple cells taken from the tumor.

According to an embodiment, the received patient information comprises other information about the cancer patient. For example, the received patient information may comprise one or more of demographic information about the patient, a diagnosis for the patient, medical history of the patient, information about the patient's tumor, and/or any other information. For example, demographic information may comprise information about the patient such as name, age, body mass index (BMI), and any other demographic information. The diagnosis for the patient may be any information about a medical diagnosis for the patient, including both historical and/or current. The medical history of the patient may be any historical admittance or discharge information, historical treatment information, historical diagnosis information, historical exam or imaging information, and/or any other information.

The patient information is received from one or a plurality of different sources. According to an embodiment, the patient information is received from, retrieved from, or otherwise obtained from an electronic medical record (EMR) database or system 270. The EMR database or system may be local or remote. The EMR database or system may be a component of the HRD score analysis system, or may be in local and/or remote communication with the HRD score analysis system.

Once the HRD score analysis system 200 receives, retrieves, or otherwise obtains the patient information from the database 270, the patient information can be utilized immediately, or may be stored in local and/or remote memory for future use in the method.

At step 130 of the method, the HRD score analysis system analyzes some or all of the received patient information to generate an HRD score for the cancer patient. The received patient information is analyzed by a trained HRD score model of the HRD score analysis system. The trained HRD score model can be any model, machine learning algorithm, classifier, or other algorithm capable of analyzing patient information to generate an HRD score. According to an embodiment, the HRD score analyzes the mRNA expression data obtained from the tumor of the cancer patient to generate an HRD score for the tumor of the cancer patient.

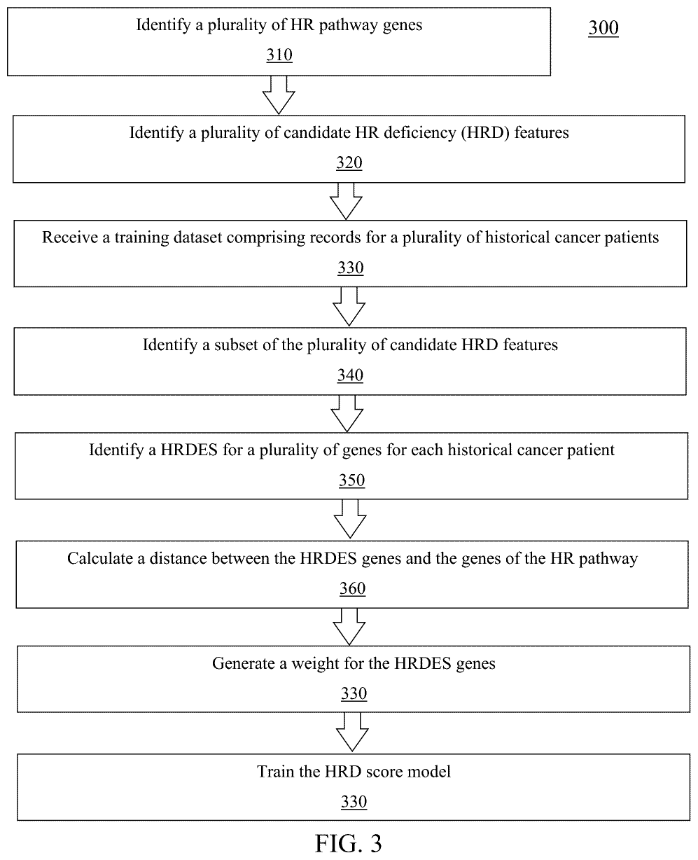

The HRD score model can be trained by a variety of mechanisms. Referring to FIG. 3, in one embodiment, is a method 300 for training an HRD score model. The HRD score model may be trained by the HRD score analysis system, or may be trained by another system and utilized by the HRD score analysis system. The trained HRD score model may be a component of or local to the HRD score analysis system, or may be remote to the system and accessed and utilized by the system remotely.

At step 310 of the method for training an HRD score model, a plurality of HR pathway genes are identified. This identification of HR pathway genes can be a manual, automated, and/or hybrid method. According to an embodiment, genes known or predicted to modulate HR pathways and/or genes known to result in mutations that cause HRD were utilized identified in step 310 of the method.

Both BRCA1 and BRCA2 are crucial for the HR pathway. BRCA1- and BRCA2-null tumors, which are deficient in HR, are thus sensitive to compounds that increase the demand on HR, such as poly ADP ribose polymerase (PARP) inhibitors. Because triple negative breast cancer (TNBC) patients with BRCA1/2 mutations have been shown to be more sensitive to chemotherapy including DNA-damaging agents (e.g. alkylating agents or anthracyline) and antimicrotubule agents), it was hypothesized that the BRCA1/2 inactivated TNBC patients would have a prolonged survival when treated with chemotherapy. As consistent with the previous studies on the effect of somatic and pathogenic germline mutations in BRCA1/2 on the survival rate in TCGA and METABRIC, TNBC patients harboring BRCA1/2 mutations showed better survival outcomes than other patients. When considering HR pathway deficiency by BRCA1/2 inactivation through other means (promoter hypermethylation, genome deletion, or transcription inhibition resulted in low expression level) in addition to gene mutations, more patients with HRD were identified and the better survival outcome in the group than others was statistically significant. Therefore, as described or otherwise envisioned herein is a defined HRD group with a favorable overall survival rate among chemotherapy-treated patients, with the overall objective of identifying TNBC patients with HRD who may benefit from PARP inhibitor and/or platinum therapy.

TNBC may have diverse defects in the HR DNA repair pathway, through mutations in other HR-pathway genes beyond BRCA1/2 such as PALB2, hypermethylation in promoter regions of HR-pathway genes such as BRCA1 and RAD51C, and other as yet to be identified mechanisms. To predict HR deficiency, the HRD score model was developed by considering all HR pathway genes in addition to BRCA1 and BRCA2. According to an embodiment, candidate genes were collected that modulate HR pathways, and candidates genes were collected that develop or otherwise have mutations that cause HRD. According to one embodiment, therefore, is a collection of genes in TABLE 1, although the plurality of HR pathway genes identified at step 310 of the method may comprise more or fewer genes than provided in TABLE 1.

TABLE 1
Identified HR Pathway Genes

| Genes | Description and Gene Function |
|---|---|
| ATM | ATM Serine/Threonine Kinase, cell cycle checkpoint kinase |
| ATR | ATR Serine/Threonine Kinase, DNA damage sensor |
| BAP1 | BRCA1 Associated Protein 1, Ubiquitin Carboxy-Terminal Hydrolase |
| BLM | BLM RecQ Like Helicase |
| BRCA I | BRCA1 DNA Repair Associated, 190 kD nuclear phosphoprotein that plays |
| | a role in maintaining genomic stability |
| BRCA2 | BRCA2 DNA Repair Associated, |
| BRIP1 | BRCA1 Interacting Helicase 1, a member of the RecQ DEAR helicase family |
| CDK12 | Cyclin Dependent Kinase 12 |
| CHEK1 | Checkpoint Kinase 1 |
| CHEIC2 | Checkpoint Kinase 2 |
| FANCA | Fanconi anemia (FA) Complementation Group A |
| FANCC | FA Complementation Group C |
| FANCD2 | FA Complementation Group D2 |
| FANCE | FA Complementation Group E |
| FANCF | FA Complementation Group F |
| MREll | MRE 1 1 Homolog, Double Strand Break Repair Nuclease |
| NBS1 (NBN) | Nibrin, associated with Nijmegen breakage syndrome, |
| PALB2 | Partner And Localizer Of BRCA2 |
| RAD50 | RAD50 Double Strand Break Repair Protein |
| RAD51B | RAD51 Paralog B |
| RAD51C | RAD51 Paralog C |
| RAD51D | RAD51 Paralog D |
| WRN | WRN RecQ Like Helicase |
| RAD54L | RAD54 Like, belongs to the DEAD-like helicase superfamily |
| FANCI | FA Complementation Group I |
| FANCL | FA Complementation Group L |
| RAD52 | RAD52 Homolog, DNA Repair Protein |
| XRCC3 | X-Ray Repair Cross Complementing 3, a member of the RecA/Rad51-related |
| | protein family |

At step 320 of the method, a plurality of candidate HR deficiency (HRD) features are identified. This identification of HRD features can be a manual, automated, and/or hybrid method. According to an embodiment, the candidate HRD features are identified using one or more of: (i) DNA mutation data; (ii) DNA copy number variation (CNV) data; (iii) DNA methylation data; and (iv) mRNA expression data to define an activity of each of the plurality of HR pathway genes.

According to an embodiment, candidate multi-omics HRD features reflecting activity status of HR pathway genes were identified. The activity status of each HR pathway gene can be determined based on its promoter methylation, expression level, CNV, and germline/somatic mutations for TCGA data, or its expression level, CNV, and somatic mutations. According to an embodiment, candidate omics-wise HRD features were selected that can represent activity status of each HR pathway gene based on each omics data as follows. By combining omics-wise HRD features of each HR pathway gene, the gene-wise HRD feature for each HR pathway gene is determined. Because omics-wise HRD features may have inconsistent association with the survival rate, omics-wise HRD features were selected that have the same direction of effect on the survival rate as a gene-wise HRD feature, resulting in 16 HRD features for the training dataset.

According to one embodiment, therefore, is a collection of HRD features in TABLE 2, although the plurality of candidate HRD features identified at step 320 of the method may comprise more or fewer candidates.

TABLE 2
Candidate HRD Features

BRCA1: Mutation status and gene expression level
BAP1: Mutation status, gene expression level, and CNV
MRE11A: Gene expression level
CHEK2: Mutation status
BLM: CNV
FANCC: Gene expression level
RAD54L: Gene expression level
WRN: CNV
BRCA2: Gene expression level
FANCA: Mutation status
ATR: Mutation status
FANCI: Gene expression level
FANCD2: Mutation status
RAD51C: Gene expression level
BRCA2: Mutation status
RAD50: Gene expression level

At step 330 of the method, a training dataset comprising records for a plurality of historical cancer patients, at least some of whom were HR deficient, is received, retrieved, or otherwise obtained. The training dataset comprises data sufficient to train the HRD score model as described or otherwise envisioned herein. According to an embodiment, therefore, the training dataset can comprise any information about the plurality of historical cancer patients that can be used to train an HRD score model, and that a trained HRD score model can utilize to generate an HRD score. According to an embodiment, the patient information comprises medical records for a plurality of historical cancer patients.

The training dataset comprising records for a plurality of historical cancer patients is received from one or a plurality of different sources. According to an embodiment, the records are received from, retrieved from, or otherwise obtained from an electronic medical record (EMR) database or system 270. The EMR database or system may be local or remote. The EMR database or system may be a component of the HRD score analysis system, or may be in local and/or remote communication with the HRD score analysis system. The received training dataset may be utilized immediately, or may be stored in local or remote storage for use in further steps of the method.

At step 340 of the method, a subset of the plurality of candidate HRD features are identified, based on an association between each of the plurality of candidate HRD features and historical cancer patient survival. According to an embodiment, the effect of each of the plurality of candidate HRD features on survival rates of the historical cancer patient training dataset was assessed using a log-rank test comparing inactivated versus activated status of the samples. According to an embodiment, samples with inactivated HR pathway based on a given selected HRD feature are defined as the HRD group. Other methods of identifying the subset of the plurality of candidate HRD features are possible. According to one embodiment, the association between overall survival associations with each of the plurality of candidate HRD features is assessed using Cox proportional hazard regression and the log-rank test.

At step 350 of the method, a plurality of HRD expression signatures (HRDES) are identified for a plurality of genes for each of a plurality of the historical cancer patients in the training dataset. According to an embodiment, the HRDES are defined by comparing gene expression between an HRD group in the training dataset and gene expression in another group in the training dataset, such as a non-HRD group. For example, gene expression can be compared between an HR pathway deficiency low group and an HR pathway deficiency high group, to define gene expression differences or changes. Gene expression changes may directly relate to HR pathway activity difference or due to downstream changes such as tumor microenvironment (TME) differences in response to HR pathway activity changes. To differentiate potential direct vs indirect changes in HRDES, the distance between each gene in HRDES was compared to the HR pathway genes in the constructed TNBC causal network.

Therefore, according to an embodiment, identifying comprises first classifying, based on the subset of candidate HRD features, the historical cancer patients into either a HRD low group or an HRD high group. Identifying further comprises comparing mRNA expression data from the HRD low group to mRNA expression data from the HRD high group. Accordingly, a subset of the plurality of candidate HRD features is identified.

At step 360 of the method, a distance is calculated between: (i) each of the plurality of genes for which an HRDES was identified and (i) one or more genes in the plurality of identified HR pathway genes, such as a constructed molecular causal network for the specific cancer type. The distance can be calculated a variety of different ways. According to an embodiment, the distance is the shortest distance found between a gene and any gene in the HR pathway network.

At step 370 of the method, a weight for each of the plurality of genes for which an HRDES was identified is generated based on the calculated distance. The weight can be calculated according to a variety of different methods, including the methods described or otherwise envisioned herein. For example, the weight for each gene can be set based on the structure of molecular causal network, such as W_{g}=e^{-d}^{_{g}}^{λ(d}^{_{g}}⁾, where d_{g} is the shortest distance of a gene g to a HR pathway gene in the network, and λ(d_{g}) is the tuning parameter. The HRD score can then be defined as a genome-wide weighted correlation between HRDES and the expression profile of a sample. Accordingly, the weighted plurality of genes can be utilized to generate an HRD score for incoming gene expression data, such as mRNA expression data obtained from a cancer patient's tumor.

At step 380 of the method, an HRD score model is trained, using the training dataset, to identify a set of final HRD features and their associated weights, and thus to generate an HRD score. The HRD score HRD score model can be any algorithm capable of being trained using the provided input, and capable of being trained to generate an HRD score. The HRD score model can be any classifier, machine learning algorithm, or any other algorithm.

Once it is trained, the HRD score model is stored in memory for subsequent analysis. The memory may be local or remote storage, and may be a component of the HRD score analysis system.

Returning to the method depicted in FIG. 1 for providing a homologous recombination DNA repair deficiency (HRD) score for a cancer patient, at step 140 of the method, the HRD score generated by the trained HRD score model of the HRD score analysis system is reported to user via a user interface. According to an embodiment, the HRD score is provided with other information about the cancer patient, including but not limited to demographic information, diagnostic or historical information about the patient or their cancer or tumor, and/or other information.

The generated HRD score can be provided using a variety of different mechanisms. For example, a text-based output or visual representation may be displayed to a medical professional or other user, including the patient, via the user interface of the system. The generated HRD score may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system or other patient analysis system such as a clinical decision support (CDS) system.

At step 150 of the method, the generated and reported HRD score is utilized by a clinician, researcher, or healthcare professional to identify and implement a treatment for the cancer patient. Specifically, the generated HRD score for the cancer patient indicates that the tumor is HR deficient, and therefore the treatment is identified and implemented to target the HR deficiency. The clinician, researcher, or healthcare professional can administer the identified HR deficiency treatment. The identified HR deficiency treatment can be any treatment that will target the HR deficiency of the tumor. According to an embodiment, the identified HR deficiency treatment is chemotherapy, immunotherapy, and/or a poly ADP ribose polymerase (PARP) inhibitor, among other possible treatments.

### Example

Described below is an example of one possible application of the methods and systems described or otherwise envisioned herein. The example is provided only as a possible embodiment of the methods and systems described or otherwise envisioned herein, and is therefore does not limit or prohibit other possible variations and embodiments. According to an embodiment, the HRD score analysis system is utilized to generate and provide an HRD score for a cancer patient's tumor.

Design and Training of an HRD Score model

Triple negative breast cancer (TNBC) may have diverse defects in the HR DNA repair pathway, through mutations in other HR-pathway genes beyond BRCA1/2 such as PALB2, hypermethylation in promoter regions of HR-pathway genes such as BRCA1 and RAD51C, and other as yet to be identified mechanisms. To predict HR deficiency, an HRD score model is developed by considering 1) all HR pathway genes in addition to BRCA1 and BRCA2; and 2) the impacts of genomic/epigenetic changes of HR pathway genes in addition to mutations. It is hypothesizes that tumors with functionally defective HR pathway genes due to genomic or epigenetic changes may be HR deficient, and may have better survival with chemotherapy and may benefit from PARP inhibitor and/or platinum treatments that target selective vulnerabilities of HR deficient tumors.

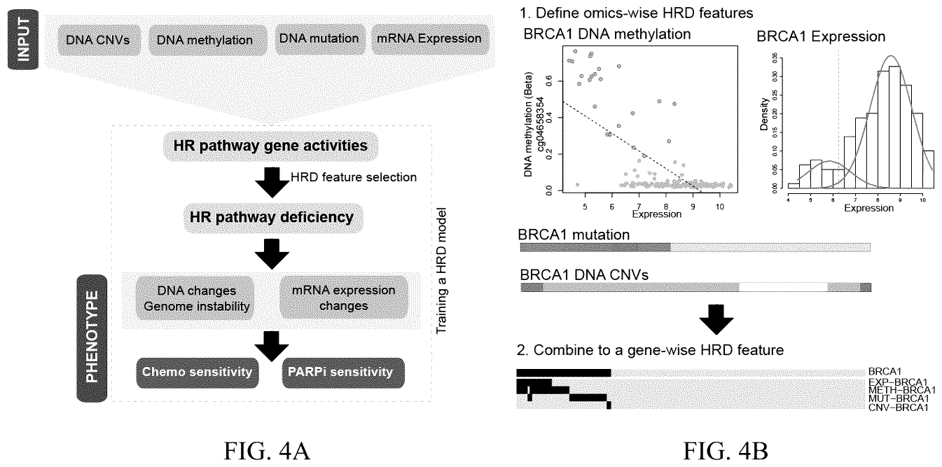

Referring to FIG. 4A, in one embodiment, is a flowchart of a method for formulating features for HR pathway deficiency predictions, as described or otherwise envisioned herein. Referring to FIG. 4B, in one embodiment, is a flowchart of a method for defining activity of HR pathway genes. In the training phase, multi-omics data was integrated (e.g. DNA mutation, DNA copy number, DNA methylation, and mRNA expression) to define activity of each HR pathway gene (FIG. 4B), referred as candidate HRD features, which could be used to formulate features for HR pathway deficiency predictions (FIG. 4A). Then, each candidate HRD feature and their combinations were evaluated in term of their association with overall survival in the training data set.

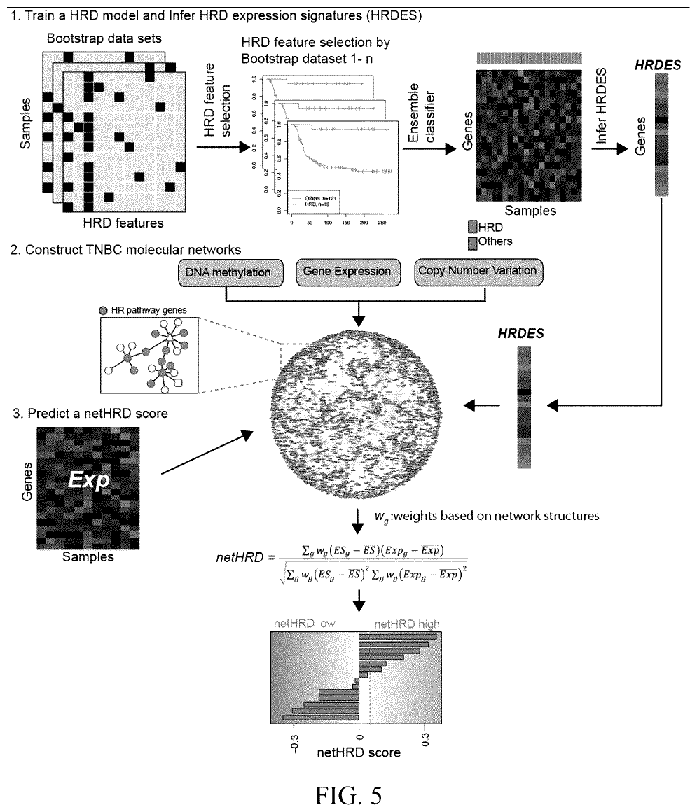

After an optimal HRD feature combination was identified, samples in the training dataset were classified into HR pathway deficiency low and high groups based on the identified HRD feature combination. Next, gene expression was compared between the HR pathway deficiency low/high groups and defined gene expression changes, referred as HRD expression signatures (HRDES). Referring to FIG. 5, in one embodiment, is a flowchart of a method for generating an HRD score model. In FIG. 5, HRDES are generated for the genes. Gene expression changes may directly relate to HR pathway activity difference or due to downstream changes such as tumor microenvironment (TME) differences in response to HR pathway activity changes. To differentiate potential direct versus indirect changes in HRDES, the distance of each gene in HRDES to the HR pathway genes in the constructed TNBC causal network was calculated. And each sample's HRD score was calculated as the similarity between its gene expression profile and HRDES through a weighted correlation with each gene's coefficient related to its distance to HR pathway genes calculated above (as shown in FIG. 5, for example. The METABRIC cohort, a TNBC cohort, has the longest follow-up time and diverse multi-omics data, thus it was used as the training dataset in this study.

HRD Scores Associate with Survival of TNBC Patients Treated with Chemotherapy

To assess the association between the HRD model trained on the METABRIC data and survival of TNBC patients from other independent cohorts, we investigated the effect of HRD scores on survival rates was investigated. For the METABRIC cohort, TNBC patients in the HRD score high group had a significantly better survival rate than the HRD score low group (log-rank p-value=0.006 as expected. For the three independent cohorts TNBC patients treated with diverse chemotherapy (see, e.g., TABLE 3), patients in the netHRD score high group consistently had a significantly better survival rate than the netHRD score low group (log-rank p-values=0.002, 0.05, and 0.002 for TCGA, GSE53752, and GSE58812, respectively). For comparison, associations of CIN70 score were assessed, which estimates genome instability, with survival in these cohorts, and the association was significant only in one cohort (log-rank p-values=0.9, 0.4, 0.04, and 0.5 for METABRIC, TCGA, GSE53752, and GSE58812, respectively). For the TCGA and METABRIC cohorts with DNA profiling data available, genomic scars by scarHRD, Signature 3 (Sig 3⁺) by Signature Multivariate Analysis (SigMA), and HRDetect were assessed in term of association with survival. Only the association of scarHRD score and survival in the TCGA cohort was significant. It is worth to note that scarHRD was developed based on TCGA data.

Referring to TABLE 3 are the results of the association with sensitivity to chemotherapy or PARP inhibitor treatment. According to the clinical responses, association was assessed using Cox proportional hazard regression or Wilcoxon rank sum test and Student's t-test.

TABLE 3
Association with sensitivity to chemotherapy or PARP inhibitor treatment

| Treatment Groups | Datasets | HRD | CIN70 |
|---|---|---|---|
| Survival after | METABRIC | 0.00456 | 0.542 |
| chemotherapy | TCGA | 0.00332 | 0.296 |
| | GSE58812 | 0.00671 | 0.937 |
| | GSE53752 | 0.0269 | 0.035 |
| Sensitivity to | GSE25066 | 0.0633, 0.113 | 0.172, 0.16 |
| Taxane | GSE106977 (Taxane) | 0.0495, 0.0496 | 0.0713, 0.0738 |
| | ISPY2: Control | 0.149, 0.183 | 0.733, 0.716 |
| | brighTNess: Arm C | 0.274, 0.262 | 0.0359, 0.0703 |
| Sensitivity to | GSE 106977 (Taxane + | 0.105, 0.115 | 0.205, 0.225 |
| Taxame + | Carboplatin) | | |
| Platinum | Mount Sinai data | 0.00932, 0.00356 | 0.093, 0.0622 |
| | brighTNess: Arm B | 6.646 × 10⁻⁴, | 0.0271, 0.0233 |
| | | 2.005 × 10⁻³ | |
| | CCLE: cisplatin v2 | 0.0292, 0.0269, | 0.3, 0.347, 0.86 |
| | | 0.0369 | |
| Sensitivity to | RIO trial | 0.00404, 1.032 × 10⁻⁴, | 0.0283, 0.0106, |
| PARP | | 9.166 × 10⁻⁵ | 0.00237 |
| inhibitor | ISPY2: DOP | 6.60 × 10⁻⁴, 0.0039 | 0.0585, 0.0191 |
| | brighTNess: Arm A | 2.637 × 10⁻⁴, | 0.00137, 0.00179 |
| | | 2.55 1 × 10⁻⁴ | |

HRD Scores Marginally Associate with Chemo-Response of TNBC Patients Treated with Taxol-AC Based Chemotherapy

The HRD score was also compared with chemo-sensitivity of TNBC patients with Taxol-AC based chemotherapy, and the association was significant only in GSE106977 (Wilcoxon p-values=0.0495, 0.0633, 0.149 and 0.274 for GSE106977, GSE25066, I-SPY 2 control arm, and BrighTNess Arm C, respectively) For comparison, the association of CIN70 scores and chemo-response was not significant in any data set.

### HRD Score Predicts Response to Platinum-Based Chemotherapy in TNBC Cell Lines and Patients

The association between the HRD score and Cisplatin sensitivity (measured as IC50) was assessed in 22 TNBC cell lines from Cancer Cell Line Encyclopedia (CCLE). The sensitivity to Cisplatin was downloaded from Genomics of Drug Sensitivity in Cancer (GDSC) project. The TNBC cell lines in the HRD score high group had significantly lower IC50 than the ones in the HRD score low group (Wilcoxon p-value=0.03). For comparison, scarHRD, CIN70, and BRCA1/2 mutation status was not associated with IC50 of Cisplatin

Next, the HRD scores were assessed with chemo-response in TNBC cohorts with platinum-based chemotherapy in neoadjuvant setting, and it was shown that the HRD scores were significantly associated with chemo-response (Wilcoxon p-values=0.10, 0.009 and 0.0007 for GSE106977, Mount Sinai cohort and Arm B in BrighTNess, respectively). For comparison, CIN70 scores were also significantly associated with chemo-response (Wilcoxon p-values=0.09 and 0.03 for Mount Sinai cohort and Arm B in BrighTNess, respectively), but CIN70 score differences between pCR and non-pCR groups were less significant than the HRD score differences were. It is worth to note that BRCA1/2 germline mutation status was not associated with platinum-based chemotherapy in the Arm B of BrighTNess trial (p-value=0.386.

HRD Score Associates with PARP Inhibitor Sensitivity in TNBC Patients Better than Existing Biomarkers

It was further investigated whether the model could predict the sensitivity for PARP inhibitors on TNBC patient samples from three clinical trials: a phase 2 window RIO clinical trial (EudraCT 2014-003319-12), a phase 2 I-SPY2 clinical trial, and a phase 3 BrighTNess clinical trial, and it was shown that the HRD scores were significantly associated with the response to PARP inhibitor (Wilcoxon p-values=0.004, 0.0006, and 0.0002 for RIO, I-SPY2, and BrighTNess Arm A, respectively). In the RIO trial, treatment naïve TNBC patients were treated with the PARP inhibitor Rucaparib for 2 weeks prior to surgery or neoadjuvant chemotherapy, and levels of circulating tumor DNA (ctDNA) were measured prior to, and at the end of treatment. Changes in ctDNA levels between baseline and end of treatment are used as a surrogate biomarker for Rucaparib response. Based on expression profiles of baseline samples, i.e. TNBC samples taken prior to Rucaparib treatment, HRD scores were measured. Notably, the HRD score is significantly associated with Rucaparib activity assessed by ctDNA changes (p-value=0.00404, 0.000103, and 9.17×10⁻⁵ based on Wilcoxon test, t-test and Spearman correlation, respectively). The HR deficiency activity is more significantly associated with Rucaparib activity than other biomarkers, HRDetect and RAD51 foci scores, used in the previous study, and CIN70 scores. In the I-SPY2 trial, the combination of PD-L1 inhibitor Durvalumab and PARP inhibitor Olaparib added to standard paclitaxel neoadjuvant chemotherapy (Durvalumab/Olaparib/Paclitaxel (DOP)) was investigated in HER2⁻ breast cancer, including 21 TNBC patients. It was detected that the HRD score is significantly associated with pCR in TNBC patients in the DOP arm (Wilcoxon test p-value=0.00066) but not significant in TNBC in the control arm who received the standard of care (Paclitaxel) (Wilcoxon test p-value=0.149). The HRD score is more significantly associated with pCR than other previously assessed biomarkers, including STAT1 cytokine signaling (STAT1_sig) and CIN70 scores that are slightly associated with pCR rates (p-value=0.148 and 0.0585 based on Wilcoxon test for STAT1_sig and CIN70, respectively. In the BrighTNess trial, TNBC patients were randomly assigned: 237 TNBC patients were treated with Paclitaxel plus Carboplatin plus Veliparib (Arm A). The HRD scores here significantly associated with the sensitivity to Veliparib (p-value=0.000264) as well as Carboplatin (p-value=0.000665), but did not associate with the sensitivity to Paclitaxel alone (p-value=0.274). The HRD score is more significant than the biomarker CIN70 previously assessed (p-value=0.00137) as well as BRCA germline mutation (p-value=1 based on Fisher's exact test). Together, these results demonstrated that the current approach has better power to identify TNBC patients who have an underlying functional defect in HR pathways and thus may sensitive to PARP inhibitor and/or platinum-based treatment.

### The Effect of PARP Inhibitor on Cell Lines is Hard to Predict

It has been shown that activities of PARP inhibitors in vitro and vivo are not consistent. HRD score and PARP inhibitor response was assessed in TNBC cell lines. Expression profiles of 22 TNBC cell lines was collected from Cancer Cell Line Encyclopedia (CCLE), and sensitivity to PARP inhibitor including Olaparib, Talazoparib, and Niraparib from Genomics of Drug Sensitivity in Cancer (GDSC) project. Based on expression profiles, netHRD scores were calculated. The HRD score was not significantly associated with IC50s, even though, cell lines with higher HRD scores consistently had lower IC50s for PARP inhibitors. It is worth noting that cell lines with BRCA1 mutations showed high IC50 values, indicating resistant to PARP inhibitor. Furthermore, application of other two HRD models, scarHRD and CIN70 scores, resulted in not significant association with PARP inhibitor sensitivity; scarHRD and CIN70 scores were positively associated with IC50 values (e.g. Niraparib). This observation indicates cell lines might not be suitable subjects to test PARP inhibitor because of the lack of tumor microenvironment components.

HRD Score Associates with TNBC Molecular Subtype

TNBC is heterogeneous and can be divided into six molecular subtypes. Previous reports show that tumors harboring BRCA1/2 mutations tend to be BL1 and BL2 TNBC types. The association between HRD scores and TNBC molecular subtypes was investigated. The HRD score was significantly lower for patients in luminal androgen receptor (LAR) subtype than ones in other subtypes. LAR cell lines were significantly more resistant to cisplatin than basal-like (BL) subtypes based on the previous study, consistent with the current results. The observations together suggest that patient of LAR subtype should be treated differently from other subtypes.

### Discussion

Herein is developed a computational framework, an HRD scoring scheme, to assess HR deficiency within TNBCs and identify HRD tumors who will benefit from PARP inhibitor therapy. Multi-omics data was integrated to define HR pathway activity, an HRD model was trained using METBRIC TNBC data with overall survival as a surrogate marker for HR deficiency, and HRD tumors were predicted by utilizing TNBC molecular causal networks. Systematic application of the trained HRD model uncovered that the HRD score consistently predicts the response to chemotherapy in 5 out of 6 independent TNBC cohorts, the response to Cisplatin in TNBC cell lines, and furthermore the response of PARP inhibitor therapy in 3 independent TNBC cohorts. In contrast, none of other existing methods for identifying HRD tumors resulted in consistent predictions of the response to chemotherapy or PARP inhibitor therapy in these TNBC cohorts.

Even though the differences between TNBC patients in the Taxol-based chemo-sensitive group in neoadjuvant (pCR groups) and TNBC in the non-pCR groups were not statistically significant, they had consistent association with pCR group having higher HRD scores, and the trend of higher HRD score associated with more likely pCR is consistent with the trend of higher HRD score associated with better overall survival of TNBC patients with taxol-based chemotherapy, suggesting clinical trials with survival benefits as endpoints are needed in addition to trials with treatment response as endpoints when evaluating treatment benefits).

It is worth noting that the association of pCR to Carboplatin/Taxol treatment with higher netHRD scores in GSE106977 is not statistically significant, but the association trend is consistent with observations in other cohorts. The treatment regimen in GSE106977 was different from the regimen Carboplatin/Taxol followed by AC in the Mount Sinai cohort and BrighTNess Arm B cohort. Doxorubicin (Adriamycin) treatment suppresses DNA damage response and in turn affects HR pathway function, which may partially explain the difference in the associations of treatment response and netHRD score in these cohorts.

The HRD score method is a flexible model that is applicable to other cancer types. In this study, the netHRD model was trained in TNBC datasets, resulting in TNBC specific HRD expression signatures, therefore, and tested in TNBC datasets to identify HRD tumors in TNBC who will benefit from PARP inhibitor. Currently PARP inhibitors have been tested in clinical trials of ovarian and breast cancer, and are FDA-approved for cancers with germline BRCA1/2 mutations. A clinical trial of Olaparib evaluated its efficacy and safety in a spectrum of BRCA1/2 germline mutations and identified that other cancer types beyond the ovarian or breast cancer could be suitable for PARP inhibitor treatment. Recent clinical trials of PARP inhibitor in prostate and pancreatic cancer have been initiated and reported. Furthermore, there is substantial ongoing investigation incorporating PAPR inhibitors into the treatment of small cell lung cancer (SCLC). A recent study detected robust HR deficient lung cancer cases among lung adenocarcinoma and lung squamous carcinoma cases, suggesting potential usage of PARP inhibitors in lung cancer. Application of the HRD model to these candidate cancer types might facilitate to predict patients who benefits from PARP inhibitor beyond BRCA1/2 germline mutations.

The HRD score more significantly associates with platinum based chemotherapy and/or PARP inhibitor sensitivity than existing biomarkers, including genomic signature based approaches such as HRDetect and scarHRD as well as CIN70 which is a mRNA signature measuring genome instability. The result suggests that the genomic signature based approaches may not accurately reflect the current HR pathway activity status in a tumor. Instead, the HRD score aims to determine HR pathway functional status of a tumor by focusing on the transcriptional changes, which may better reflect the dynamical change in HR pathway functional status.

In summary, it is shown that the HRD score is significantly associated with platinum-based chemotherapy responses as well as PARP inhibitor treatment responses in multiple TNBC cohorts. The HRD model was compared with existing models for predicting HR deficiency, and the HRD model consistently performed better than commonly used methods. The HRD score can identify additional TNBC patients with HRD who carry wildtype BRCA1/2. The findings demonstrate that the HRD score can be a predictive biomarker for identifying TNBC patients in addition to BRCA1/2 germline mutation who may respond to platinum-based chemotherapy and/or PARP inhibitor treatments.

### Materials and Methods

The Cancer Genome Atlas (TCGA) data. Multi-omics profiles of breast cancer data from TCGA were downloaded from Genomic Data Commons (GDC) data portal. For mRNA expression data, mapped and gene-level-summarized (level 3, Reads Per Kilobase of transcript per Million mapped reads (RPKM)) RNA-seq profiles were downloaded. Log 2 transformation was performed after adding a count of 1 to each value. The log 2 transformed values were used for further analysis. For DNA methylation data, level 3 data was downloaded, ß value, measured in HM450 platform and HM27 platform. For somatic mutation data, variant calls (i.e. VCF formatted file) processed by VarScan2 downloaded. For germline mutation data, pathogenic germline variant calls of TCGA patients from the previous study downloaded. For copy number variation data, numeric focal-level Copy Number Variation (CNV) values generated by using GISTIC2 downloaded. Allelic imbalance profiles inferred from GenomeWideSNP6 Affymetrix array by using ASCAT were downloaded. Triple negative breast cancer (TNBC) is characterized by lack of expression of estrogen receptor (ER), progesterone receptor (PR) and human epidermal growth factor receptor type 2 (HER2).

Molecular Taxonomy of Breast Cancer International Consortium (METABRIC) Data: The METABRIC data breast cancer dataset was downloaded through the European Genome-Phenome Archive (study id EGAS00000000083) and consists of 1904 breast tumors, including 290 TNBC with matching detailed clinical annotations, long-term follow-up, expression data, and CNV data. The mRNA expression was profiled using Illumina HT-12 v3 platforms. The normalized mRNA expression data was downloaded and used for further analysis. For CNV data, CNV values were measured by Affymetrix SNP 6.0 and derived by using the circular binary segmentation (CBS) algorithm implemented in the DNAcopy Bioconductor package. Allelic imbalance profiles inferred from Affymetrix SNP 6.0 data by using ASCAT were downloaded. The somatic mutation data was downloaded from a previous study, which measured somatic mutation profiles for 173 of the most frequently mutated breast cancer genes by targeted sequencing. Among 173 breast cancer genes, 8 are HR pathway genes, including BRCA1 and BRCA2. The clinical outcomes were grouped into four categories according to the cause of death: alive, dead of breast cancer, dead of other causes, and dead of unknown causes. The death of other causes and unknown causes were treated as censored in survival analysis. Among 290 TNBC tumors, 140 tumors of TNBC patients with chemotherapy treatment were used for further analysis.

Other chemotherapy treated TNBC datasets with gene expression profiles: Publicly available TNBC datasets were searched that 1) have gene expression profiles available, 2) have chemotherapy treatment information, 3) have clinical outcomes such as overall survival or chemo-sensitivity measurements (e.g. pathologic complete response (pCR)), and 4) consist of more than 50 samples. Four independent TNBC datasets were identified and downloaded from Gene Expression Omnibus (GEO), of which accession numbers are GSE25066, GSE106977, GSE58812, and GSE53752. Pathologic complete response (pCR) and/or residual cancer burden were used as clinical outcomes for the datasets (GSE25066 and GSE106977) of samples with neoadjuvant chemotherapy treatment. Otherwise, overall survival rates or metastasis free survival rates were used as clinical outcomes. For GSE25066 dataset, following the definition of the previous study, chemo-sensitive groups were defined as samples showing pCR or minimal residual cancer burden (RCB-I) and resistant groups were defined as samples showing extensive residual cancer burden (RCB-II/III). For GSE106977 dataset, chemo-sensitive groups were defined as samples showing pCR.

TNBC Cell line data: RNA-seq profiles of 1019 human cancer cell lines were downloaded from Cancer Cell Line Encyclopedia (CCLE) at the CCLE portal, including expression profiles of 22 TNBC cell lines. Drug sensitivity data (i.e. Half-maximal inhibitory concentration (IC50)) was also downloaded for cancer cell lines from Genomics of Drug Sensitivity in Cancer (GDSC) project, including sensitivity to Cisplatin and PARP inhibitors, i.e. Olaparib, Talazoparib, and Niraparib. Following the recommendations from GDSC project, a second version of GDSC data set (GDSC2) was used because GDSC2 has been screened using improved equipment and procedures. Raw Affymetrix SNP6.0 arrays CEL files of CCLE project were downloaded from depmap portal to determine allelic imbalance profiles (see section Genomic Scar by scarHRD).

PARP inhibitor treated TNBC cohorts with gene expression profiles: three TNBC datasets treated with PARP inhibitors with gene expression profiles were downloaded.

RIO trial data: RNA-seq profiles of TNBC patients treated with the PARP inhibitor Rucaparib were downloaded from the European Genome-phenome Archive (EGA), reference EGAS00001004405. RNA-seq profiles include 20 paired tumor samples taken prior to, and at the end of treatment. Sequencing reads in fastq files were aligned to the GRCh37 genome using STAR aligner. Gene counts were quantified using featureCounts in Rsubread package of R. Gene count values were normalized to trimmed mean of M values (TMM) by using edgeR package in R. Changes in circulating tumour DNA (ctDNA) counts reported in FIG. 4 of the previous study were used as clinical outcomes. Other biomarkers including HRDetect and RAD51 foci deficiency assessed in the previous study were reported.

I-SPY2 (Investigation of Series studies of Predictive Your therapeutic response with imaging and molecular analysis 2) trial data: Expression profiles of 105 HER2⁻ patients treated with Durvalumab plus Olaparib in the phase II I-SPY2 trial were downloaded from GEO (accession number GSE173839). This trial consists of 71 HER2⁻ patients (including 21 TNBC patients) on the durvalumab/olaparib arm and 34 HER2⁻ patients (including 19 TNBC patients) on the control arm. Pathologic complete response (pCR) is used as clinical outcomes with neoadjuvant treatment. Other predictive gene expression biomarkers such as STAT1 cytokine signaling (STAT1_sig) and a DNA repair deficiency signature (PARPi7) assessed in the previous study were also downloaded for comparison.

BrighTNess trial data: RNA-seq profiles of TNBC patients in a phase 3, randomized, double-blind, placebo-controlled trial, BrighTNess, were downloaded from GEO (accession number GSE164458). This trial consists of TNBC patients to receive the addition of the PARP inhibitor Veliparib plus Carboplatin to standard neoadjuvant chemotherapy (Arm A, n=237), or the addition of Carboplatin to standard neoadjuvant chemotherapy (Arm B, n=122), or standard neoadjuvant chemotherapy with Paclitaxel followed by Doxorubicin/Cyclophosphamid (Arm C, n=123). Pathologic complete response (pCR) is used as clinical outcomes.

### RNA Sequencing

Preprocessing of RNA-seq profiles: Samples were aligned to the GRCh37 genome using STAR aligner. Gene counts were established using featureCounts. DeSeq2 was used to establish gene-wise normalization.

### Collecting HR Pathway Genes

Candidate genes that modulate HR pathways were collected based on a BRCAness review paper. Additional genes were added additional genes whose mutations cause HRD, resulting in total 29 HR pathway genes listed in TABLE 1.

Determining candidate functional mutations in HR pathway genes: For downloaded variant calls in the training dataset, all silent mutations were removed. Additionally, plausible non-functional mutations were removed as follows. Mutations in intron were removed except the mutations that are associated with large expression level changes (the expression levels of samples with mutations were higher than the upper quartile or lower than the lower quartile.

Candidate multi-omics HRD features reflecting activity status of HR pathway genes: The activity status of each HR pathway gene is determined based on its promoter methylation, expression level, CNV, and germline/somatic mutations for TCGA data, or its expression level, CNV, and somatic mutations for METABRIC data. Candidate omics-wise HRD features were selected that can represent activity status of each HR pathway gene based on each omics data as follows.

For DNA methylation data of TCGA, inactivated status was defined based on promoter methylation level for cis-methyl HR pathway genes. To determine cis-methyl genes, a linear relationship was assessed as follows: Exp_{g}˜Methyl_{g} where Exp_{g} indicates the expression level of gene g, Methyl_{g} indicates the DNA methylation level in the gene g's promoter region. Cis-methyl genes were defined as genes with a significant negative coefficient for Methyl_{g} variable at false discovery rate (FDR) 1% corresponding to p-value <1×10⁻⁸. In the case of multiple probes mapping to the same gene, the probe was selected with the smallest p-value. Two cis-methyl HR pathway genes were identified, BRCA1 and RAD51C. For these two cis-methyl HR pathway genes, samples with inactivated status were determined. Because there are multiple probes mapping to BRCA1, samples with inactivated status were determined by using hierarchical clustering based on methylation levels of all probes mapping to BRCA1.

For gene expression data in the training dataset, it was first investigated whether expression levels of each gene follow one or a mixture of two normal distributions. For each gene, a mixture of two normal distributions was fit to expression levels of each gene based on estimated parameters by expectation-maximization (EM) algorithm (use normalmixEM function of mixtools package in R), and calculated Bayesian information criterion (BIC). BIC for each gene based on fitting a mixture was compared to that calculated based on fitting one normal distribution. HR pathway genes were identified whose expression levels were more likely arose from a mixture of two normal distributions (lower BIC value based on fitting a mixture than that based on fitting one normal distribution), resulting in 12 HR pathway genes. For these 12 HR pathway genes whose expression levels were more likely arose from a mixture of two normal distributions, inactivated samples were determined by calculating the posterior probability of the expression level of each sample to have been generated from one of two normal distribution with the lower mean. Inactivated samples were defined as the one whose posterior probability is bigger than 0.9 for each gene, and determine the threshold for each gene.

For CNV data of TCGA and METABRIC, a tumor with homozygous deletion (i.e. value =−2) within the gene coding region was defined as the inactivated sample for the corresponding gene. For somatic mutations of TCGA and METABRIC, a tumor with at least one candidate functional somatic mutation within the gene region was defined as the inactivated sample.

By combining omics-wise HRD features of each HR pathway gene, the gene-wise HRD feature for each HR pathway gene was determined. Because omics-wise HRD features may have inconsistent association with the survival rate omics-wise HRD features were selected that have the same direction of effect on the survival rate as a gene-wise HRD feature, resulting in 16 HRD features for METABRIC, the training dataset, as shown in TABLE 2.

### Procedure to Aggregate Multi-Omics HRD Features to Define HRD Group

The aim is to define a HRD group with a favorable survival rate by using a stepwise selection procedure for candidate HRD features (i.e. omics/gene-wise inactivation status of HR pathway genes) as follows. First, the candidate HRD features with the most significant effect on survival rates were selected, which is assessed using the log-rank test comparing inactivated versus activated status. The samples with inactivated HR pathway based on a given selected HRD feature are defined as the HRD group. Next, among the rest HRD features that are not selected in the previous step, additional individual HRD features were selected. For each of HRD features that has not been selected in the previous steps, HRD samples defined from the previous step were aggregated with inactivated samples based on the given HRD feature, and then assess the significance of the survival difference between aggregated HRD samples vs. others. Then, the HRD feature resulting in an aggregated HRD group that has the most significant favorable survival rate compared to the rest of samples, is selected. Next, the HRD group is updated by adding samples with inactivated HR pathway activity based on the selected HRD feature. This iterative procedure was performed until the significance of survival association does not improve compared to the previous step. This procedure results in a HRD group with the most favorable survival rate compared to the rest of tumors.

### Integration of Multi-Omics Data to Construct a Molecular Causal Network Model for TNBC

TNBC specific causal networks were constructed based on genomic (i.e. copy number variation), epigenetic (i.e. DNA methylation) and transcriptomic data of the TCGA TNBC dataset by using Reconstructing Integrative Molecular Bayesian Networks (RIMBANet), which statistically infers causal relationships between gene expression, protein expression and clinical features that are scored in hundreds of individuals or more. In total, 9612 informative genes (mean >5.17 and variation >0.39, each corresponding to 30% quantiles of mean and 25% quantiles of variation) were included in the network reconstruction process. Cis-CNV and cis-methyl data was incorporated as priors such that cis-CNV/cis-methyl were parent nodes of the corresponding genes with cis-CNV/cis-methyl. Integrating genetic/genomic data such as cis-methyl/cis-CNV improves the quality of the network reconstruction by simulation and by experimental validations. Cis-CNV and cis-methyl was identified as follows.

Identifying cis-CNV. To identify cis-acting CNV on its own expression levels, a linear regression model was used for CNV and mRNA expression level of each gene: Exp_{g}˜CNV_{g} where indicates the expression level of a gene g, CNV_{g} indicates CNV for a gene g. Cis-acting CNV were defined as the CNV which positively associates with the corresponding gene's mRNA expression level with a stringent p-value <1×10⁻¹⁰ (corresponding to FDR 3.5×10⁻¹⁰). At p-value <1×10⁻¹⁰, 1368 cis-CNV genes were identified in the TCGA TNBC dataset.

Identifying cis-methyl. To determine cis-methyl genes, a linear regression model was applied as follows: Exp_{g}˜Methyl_{g} where Exp_{g} indicates the expression level of a gene g, Methyl_{g} indicates the DNA methylation level in a gene g's promoter region. Cis-methyl genes were defined as genes with a significant (p-value <1×10⁻¹⁰) negative coefficient for Methyl_{g} variable. In the case of multiple probes mapping to the same gene, the probes with the best p-value were selected. A total of 514 cis-methyl genes for the TCGA TNBC dataset were identified.

### Training a Network-Based HRD Score (HRD)

First, based on a training dataset, HRD tumors were identified following the HRD feature selection procedure described above. To avoid overfitting and identify robust HRD features, a bootstrap aggregating (i.e. bagging) procedure was implemented on selection of HRD features. For each bootstrapped dataset, a set of HRD features were selected (see the above section), and aggregate the selected features from each bootstrap dataset to define the ensemble classifier. The training procedure was applied to METABRIC TNBC dataset and identified four robust HRD features (BRCA1:Mut-Exp, BAP1:Mut-Exp-CNV, CHECK2:Mut, and FANCC:Exp), the HRD group was defined as the union of samples of inactivated status based on at least one of the four selected features. Secondly, HRD Expression Signatures (HRDES) representing the genome wide expression changes between the HRD tumors vs. the other tumors were inferred by fitting a linear model: Exp_{g}=β_{g}λHRD, where Exp_{g} indicates the expression levels of a gene g, HRD indicates whether a sample is assigned as a HRD group. After this analysis is completed for all genes, the HRDES is a vector of the regression coefficient β across all genes. Finally, the constructed TNBC molecular network was leveraged to distinguish gene expression changes likely due to direct or indirect effects of HRD pathway inactivation and infer a HRD score of a sample based on its expression profile. The HRD score is defined a genome-wide weighted correlation between HRDES and the expression profile of a sample:

$$netHRD score = \frac{{\sum }_{g} {w}_{g} ( {ES}_{g} - \overline{ES} ) ( {Exp}_{g} - \overline{Exp} )}{\sqrt{{\sum }_{g} {{w}_{g} ( {ES}_{g} - \overline{ES} )}^{2} {\sum }_{g} {{w}_{g} ( {Exp}_{g} - \overline{Exp} )}^{2}}} \qquad ( Eq . 1 )$$

where ES_{g} indicates the HRDES value for a gene g, Exp_{g} indicates the expression level of a gene g in the sample, and W_{g} indicates the weight of a gene g. The weight for each gene is set based on the structure of molecular causal network of TNBC as W_{g}=e^{-d}^{_{g}}^{λ(d}^{_{g}}⁾, where d_{g} is the shortest distance of a gene g to a HR pathway gene in the network, and λ(d_{g}) is the tuning parameter. If λ is set to 0, then all genes in the network have the same weight for all genes.

### Determining HRD High and Low Groups Based on HRD Scores

Based on HRDES inferred in the training dataset, METABRIC, HRD scores were calculated for the METABRIC samples. As expected, the HRD group (i.e. samples with inactivated HR pathway activity based on at least one of selected HRD features) has higher HRD scores compared to others. The threshold was determined as a lower limit of 90% confidence interval based on HRD scores of HRD group, and re-assigned HRD samples whose scores are higher than the threshold. Then, the threshold is used to define HRD samples for other testing datasets.

### Inferring Gene Activities Based on Gene Expression and TNBC Network

In general, it is assumes that a gene's activity correlate with its expression level. However, the expression of BRCA1 is not always a good surrogate of its activity. Some of tumors with BRCA1 mutations have a high BRCA1 expression level. Because the HRD scoring procedure is dependent on the BRCA1 expression, BRCA1 expression was inferred based on TNBC molecular structure to reduce noises in the HRD prediction phase.

Association with Clinical Outcomes

The association between the overall survival associations with the predicted HRD score for datasets with available overall survival information (i.e. TCGA, METABRIC, GSE58812, GSE53752) was analyzed using Cox proportional hazard regression and the log-rank test. For the datasets with clinical outcomes pCR and/or RCB (i.e. GSE25066, GSE106977, Mount Sinai TNBC cohort, I-SPY2 trial, RIO trial, and BrighTNess trial), the association between the predicted HRD scores and chemo-sensitive/resistant group was assessed using Wilcoxon rank sum test and Student's t-test. In cases where the sensitivity measurements (i.e. IC50 for CCLE and ctDNA changes for RIO trial) are available, the p-value was calculated based on Spearman's rank correlation coefficient.

### Application of Other Methods

The following four previous biomarkers, i.e. Genomic scars, Signature 3, HRDetect, CIN70 genomic instability scores, were investigated for the performance comparison.

Genomic scars by scarHRD: The three genomic scars were determined for three datasets with SNP arrays, including TCGA, METABRIC and CCLE data. Allelic imbalance profiles were downloaded for TCGA and METABRIC data, or generated using ASCAT for CCLE to determine the scores for the three genomic scars, the number of telomeric allelic imbalances (NtAI), homologous recombination deficiency loss of heterozygosity score (HRD-LOH), and large scale transition (LST). For CCLE data, raw Affymetrix SNP6.0 arrays CEL files were processed using an R-package “Rawcopy” to create the probe level log 2 ratio (log R) signal, and B-allele frequency (BAF) signal. These data were inputs into the ASCAT algorithm. Three genomics scar scores (i.e. NtAI, LST, HRD-LOH) were determined by allelic imbalance profiles using the scarHRD R package. The combined HRD score was derived from these three independent genomic scars. A myChoice HRD threshold of 42 has previously been developed to identify HRD tumors using this test. As following the previous study, tumors are considered HRD⁺ if they have a high combined HRD score (≥42).

Signature 3 by Signature Multivariate Analysis (SigMA): One of mutational signatures, ‘Signature 3 (Sig3)’, corresponds to a deficiency in the HR machinery. Sig3 was investigated for two datasets with mutation profiles, TCGA and METABRIC data. In particular, a computational tool called SigMA was used because SigMA is not limited to whole-genome data and but can be used to whole exome data (TCGA data) and targeted sequencing panels (METABRIC data).

HRDetect: The HRDetect algorithm was applied to TCGA data. HRDetect scores were investigated using whole exome sequencing (WES) data and allelic imbalance profiles inferred from GenomeWideSNP6 Affymetrix array data. As the number of mutations significantly reduced in WES versus WGS and rearrangement signatures were not available for WES data, the algorithm was re-trained using WES based data as the input. Following the description of the methods that were used in the original HRDetect model, the information on signatures of single base substitutions, indels, and copy number classification was utilized based on HRD indices as the predictor variables in the training of HRDetect algorithm. Each predictor variable was generated as follows.

HRD indices: HRD index was calculated as an HRD-LOH score inferred using scarHRD (see the section Genomic scars by scarHRD), and used as a input in to the algorithms.

Substitution signatures: Landscape of somatic substitution signatures were extracted with deconstructSigs R packages based on vcf files downloaded from GDC data portal by using the COSMIC signatures database as a mutational-process matrix. After the evaluation of their signature compositions, the mutational catalogs of the samples were reconstructed, and the cosine of the angle between the 96-dimensional original and reconstructed vectors were measured. Samples whose cosine similarities were smaller than 0.8 were considered non-reconstructable, and were removed from any further analysis. Counts of mutations associated with each signature of substitutions signatures 1, 2, 3, 5, 6, 8, 13, 17, 18, 20, 26 were used as inputs into the algorithms.

Indel signatures: Indel signatures were extracted using MutationalPatterns R packages. The number of insertions, number of repeats, number of ≥3 microhomologies, and number of unique deletions were extracted and were used as inputs into the algorithms.

Fitting a LASSO logistic regression: Following the methods that were used in the original HRDetect model, the predictor variables were log-transformed and standardized. A lasso logistic regression model was used to separate the two categories of patient samples: those affected or not affected by BRCA1/BRCA2 mutants by using glmnet R packages. The value of the regulatory parameter λ was determined by examining 300 runs of independent tenfold nested cross validation training.

genomic instability scores: The measure of chromosomal instability (CIN70) was investigated as previously described: 70 top-ranked genes with the highest CIN score were collected and CIN70 score was predicted by calculating the mean of the ranks of each gene.

TNBC molecular subtype: was determined using the TNBCtype tool after normalization.

False discovery rate (FDR): To calculate FDR rates based on p-value, p.adjust function in R with Benjamini and Hochberg method was used.

Referring to FIG. 2 is a schematic representation of an HRD score analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record system 270 is an electronic medical records database from which the information about a plurality of patients, including demographic, diagnosis, and/or treatment information may be obtained or received. According to an embodiment, the electronic medical record system 270 is an electronic medical records database from which the training data utilized to train the HRD score model. The training data can be any data that will be utilized to train the algorithm. The training data can comprise any other information. The electronic medical records database may be a local or remote database and is in direct and/or indirect communication with system 200. Thus, according to an embodiment, the system comprises an electronic medical record database or system 270.

According to an embodiment, storage 260 of system 200 may store one or more algorithms, modules, and/or instructions to carry out one or more functions or steps of the methods described or otherwise envisioned herein. For example, the system may comprise, among other instructions or data, HRD score model training instructions 262, a trained HRD score model 263, and/or reporting instructions 264.

According to an embodiment, HRD score model training instructions 262 direct the system to train a model to be an HRD score model. The HRD score model may be trained by the HRD score analysis system, or may be trained by another system and utilized by the HRD score analysis system. The trained HRD score model may be a component of or local to the HRD score analysis system, or may be remote to the system and accessed and utilized by the system remotely. Referring to FIG. 3, in one embodiment, is an example method for training an HRD score model, and thus the HRD score model training instructions 262 can direct the system to train the HRD score model as described with regard to FIG. 3.

According to an embodiment, the system comprises a trained HRD score model 263. The trained model can be any algorithm, classifier, or model capable of creating the output, including but not limited to machine learning algorithms, classifiers, and other algorithms. The trained algorithm is a unique algorithm based on the training data used to train the algorithm. Once generated, the trained algorithm can be utilized or deployed immediately, or it may be stored in local and/or remote memory for future use and/or deployment. Thus, the system comprises a trained HRD score model 263 configured to generate the HRD score for a subject as described or otherwise envisioned herein.

According to an embodiment, reporting instructions 264 direct the system to direct the system to generate and provide to a user via a user interface information comprising the HRD score generated by the trained HRD score model 263. The information may be communicated by wired and/or wireless communication to another device. For example, the system may communicate the information to a mobile phone, computer, laptop, wearable device, and/or any other device configured to allow display and/or other communication of the information.

According to an embodiment, the HRD score analysis system is configured to process many thousands or millions of datapoints in the input data used to train the HRD score algorithm, as well as to process and analyze the vast plurality of input data. For example, generating a functional and skilled trained HRD score algorithm using an automated process such as feature identification and extraction and subsequent training requires processing of millions of datapoints from input data and the generated features. This can require millions or billions of calculations to generate a novel trained HRD score algorithm from those millions of datapoints and millions or billions of calculations. As a result, each trained HRD score algorithm is novel and distinct based on the input data and parameters of the machine learning algorithm, and thus improves the functioning of the HRD score analysis system. Thus, generating a functional and skilled trained HRD score algorithm comprises a process with a volume of calculation and analysis that a human brain cannot accomplish in a lifetime, or multiple lifetimes. By providing an improved analysis system for a patient using the HRD score algorithm as described or otherwise envisioned herein, this novel HRD score analysis system has an enormous positive effect on patient analysis and care compared to prior art systems.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method (100) for providing a homologous recombination DNA repair deficiency (HRD) score for a cancer patient, comprising:
receiving (120) information about the cancer patient, the information comprising at least mRNA expression data obtained from a tumor of the cancer patient;
analyzing (130), using a trained HRD score model, the received information about the cancer patient to generate an HRD score for the cancer patient; and
providing (140), via a user interface, the generated HRD score for the cancer patient;
wherein the HRD score model is trained by:
(i) Identifying (310) a plurality of HR pathway genes;
(ii) generating (320) a plurality of candidate HR deficiency (HRD) features using (i) DNA mutation data; (ii) DNA copy number variation (CNV) data; (iii) DNA methylation data; and (iv) mRNA expression data to define an activity of each of the plurality of HR pathway genes;
(iii) receiving (330) a training dataset comprising records for a plurality of historical cancer patients, at least some of whom were HR deficient;
(iv) determining (340), using the training dataset, a subset of candidate HRD features based on an association between each of the plurality of candidate HRD features and historical cancer patient survival;
(v) identifying (350) HRD expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group;
(vi) calculating (360), for each of the plurality of genes for which a HRDES was identified, a distance between the gene and a plurality of HR pathway genes within a constructed molecular causal network for the cancer type;
(vii) weighting (370), based on the calculated distance, one or more of the plurality of genes in the HRDES; and
(viii) training (380), using training dataset, the HRD score model to identify a set of final HRD features and their associated weights.

2. The method of claim 1, wherein the generated HRD score for the cancer patient indicates that the tumor is HR deficient.

3. The method of claim 2, further comprising the step of implementing (150), when the generated HRD score for the cancer patient indicates that the tumor is HR deficient, a treatment to target the HR deficiency.

4. The method of claim 3, wherein the treatment to target the HR deficiency is chemotherapy, and/or a poly ADP ribose polymerase (PARP) inhibitor.

5. The method of claim 1, wherein the set of final HRD features comprises one or more of the genes in TABLE 1.

6. A method (100) for treating a cancer patient, comprising:
receiving (140) a generated HRD score for the cancer patient indicating that the tumor is HR deficient; and
administering (150) a treatment to the cancer patient;
wherein the HRD score is generated by:
receiving (120) information about the cancer patient, the information comprising at least mRNA expression data obtained from a tumor of the cancer patient;
analyzing (130), using a trained HRD score model, the received information about the cancer patient to generate an HRD score for the cancer patient;
wherein the HRD score model is trained by:
(i) identifying (310) a plurality of HR pathway genes;
(ii) generating (320) a plurality of candidate HR deficiency (HRD) features using (i) DNA mutation data; (ii) DNA copy number variation (CNV) data; (iii) DNA methylation data; and (iv) mRNA expression data to define an activity of each of the plurality of HR pathway genes;
(iii) receiving (330) a training dataset comprising records for a plurality of historical cancer patients, at least some of whom were HR deficient;
(iv) determining (340), using the training dataset, a subset of candidate HRD features based on an association between each of the plurality of candidate HRD features and historical cancer patient survival;
(v) identifying (350) HDR expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group;
(vi) calculating (360), for each of the plurality of genes for which a HRDES was identified, a distance between the gene and a plurality of HR pathway genes within a constructed molecular causal network for the cancer type;
(vii) weighting (370), based on the calculated distance, one or more of the plurality of genes in the HRDES is utilized to generate an HR score;
(viii) training (380), using training dataset the HR score model to identify a set of final HRD features and their associated weights.

7. The method of claim 7, wherein the treatment is chemotherapy, and/or a poly ADP ribose polymerase (PARP) inhibitor.

8. The method of claim 7, wherein the set of final HRD features comprises one or more of the genes in TABLE 1.

9. The method of claim 1, wherein the subject has been diagnosed with cancer, is at risk of having cancer, or is suspected of having cancer.

10. The method claim 1, wherein the cancer is selected from the group consisting of triple negative breast cancer, human epidermal growth factor receptor 2-negative breast cancer, estrogen receptor-dependent breast cancer, ovarian cancer, prostate cancer, lung cancer, colorectal cancer, and/or other solid cancer, leukemia, lymphoma and/or other blood cell cancer, and any combination thereof.

11. A system (200) configured to provide a homologous recombination DNA repair deficiency (HRD) score for a cancer patient, comprising:
information about the cancer patient, the information comprising at least mRNA expression data obtained from a tumor of the breast cancer patient;
a trained HRD score model (262);
a processor (220) configured to analyze, using the trained HRD score model, the received information about the cancer patient to generate an HRD score for the cancer patient; and
a user interface (240) configured to provide the generated HRD score for the cancer patient;
wherein the HRD score model is trained by:
(i) identifying a plurality of HR pathway genes;
(ii) generating a plurality of candidate HR deficiency (HRD) features using (i) DNA mutation data; (ii) DNA copy number variation (CNV) data; (iii) DNA methylation data; and (iv) mRNA expression data to define an activity of each of the plurality of HR pathway genes;
(iii) receiving a training dataset comprising records for a plurality of historical cancer patients, at least some of whom were HR deficient;
(iv) determining, using the training dataset, a subset of candidate HRD features based on an association between each of the plurality of candidate HRD features and historical cancer patient survival;
(v) identifying HDR expression signatures (HRDES) for a plurality of genes for each of a plurality of the historical cancer patients in the training dataset, wherein identifying comprises: (a) classifying, based on the subset of candidate HRD features, the historical cancer patients into either a HRD low group or an HRD high group; and (b) comparing mRNA expression data from the HRD low group to mRNA expression data from the HRD high group;
(vi) calculating, for each of the plurality of genes for which a HRDES was identified, a distance between the gene and a plurality of HR pathway genes within a constructed molecular causal network for the cancer type;
(vii) weighting, based on the calculated distance, one or more of the plurality of genes in the HRDES is utilized to generate an HR score; and
(viii) training, using training dataset the HR score model to identify a set of final HRD features and their associated weights.

12. The system of claim 11, wherein the generated HR score for the cancer patient indicates that the tumor is HRD deficient.

13. The system of claim 12, wherein the system is further configured to recommend, when the generated HRD score for the cancer patient indicates that the tumor is HR deficient, a treatment to target the HR deficiency.

14. The system of claim 13, wherein the treatment to target the HR deficiency is chemotherapy, and/or a poly ADP ribose polymerase (PARP) inhibitor.

15. The system of claim 11, wherein the set of final HRD features comprises one or more of the genes in TABLE 1.
